(19) United States
(12) Patent Application Publication
(10) Pub. No.: US 2005/0244316 A1
(43) Pub. Date: Nov. 3, 2005

(54) Corner protector for preventing tearing of sterilization wrap wrapped around a sterilization tray

(76) Inventor: Phillip Davis, Weston, CT (US)

Correspondence Address: EDWARDS & ANGELL, LLP, P.O. BOX 55874, BOSTON, MA, 02205, US

(21) Appl. No.: 10/442,220

(22) Filed: May 20, 2003

(51) Int. Cl. A61L 2/00

(52) U.S. Cl. 422/300

(57) ABSTRACT

A sterilization wrap protector separates a sterilization tray from a sterilization wrap, the protector may include a foam plastic material having an inside engagement surface. The inside engagement surface may include a first inside surface that is generally planar, a second inside surface which extends from the first inside surface and that is generally arcuate in cross section and a third inside surface which also extends from the first inside surface and is also generally arcuate in cross section. An outside engagement surface may include a first outside surface that is generally planar, a second outside surface that is generally planar and which extends in a direction that is generally perpendicular to the first outside surface and a third outside surface that is generally planar and which extends in a direction that is generally perpendicular to the first outside surface and to the second outside surface.

### BACKGROUND

1. Technical Field

The present invention relates to devices for maintaining sterilization and, more particularly, to devices for preventing rips or tears in a sterilization wrap wrapped about a sterilization tray.

2. Related Art

Sterilization of surgical tools including scalpels, scissors and clamps may be accomplished in a known manner by placing the tools in a sterilization tray, wrapping the tray with a sterilization wrap to form a sterilization pack and then placing the pack into a sterilizer. Once a sterilization pack has completed a sterilization cycle, it may be removed from the sterilizer and stored for future use during surgery.

One long and continuing problem encountered with sterilization packs includes tearing and ripping open of the sterilization wrap prior to use of the sterilized surgical equipment. Of course, once a pack is torn, the entire contents are deemed contaminated with bacteria and the like and, therefore, must be re-wrapped and then re-sterilized. A typical location where a rip may occur is at a corner of a sterilization pack.

One prior art corner protector for preventing rips in a sterilization wrap of a sterilization pack is shown at 100 in FIG. 1. The corner protector 100 is composed of reticulated plastic foam and has a rectangular, block-like shape. A perforation 102 is provided to divide the corner protector 100 into two pairs of tab portions 104 and 106 each of which may be folded along dashed lines 105 and 107.

As shown in FIG. 2, the corner protector 100 may be attached to a corner edge 108 of a sterilization tray 110 whereby each of the pairs of tab portions 104 and 106 may be folded along the dashed lines 105 and 107.

Several problems arise in connection with use of the corner protector 100. For example, the corner protector 100 fails to completely cover the sharp corner edge points 112 and thus rips, in a sterilization wrap (see FIG. 4) wrapped over the corner protector, regularly occur. Also, the corner protector 100 tends to slip off the corner 108 during assembly making it difficult for a sterilization wrap to be wrapped over the corner protector.

What is needed, therefore, is a more effective corner protector that will better prevent rips and tears in a sterilization wrap and is more easily attached to a sterilization tray.

### SUMMARY OF THE DISCLOSURE

In accordance with an aspect of the present invention, a sterilization wrap protector, that may separate a sterilization tray from a sterilization wrap, comprises a foam plastic material that includes an inside engagement surface. The inside engagement surface may comprise a first inside surface that is generally planar, a second inside surface that is generally planar and which extends in a direction that is generally perpendicular to the first inside surface and a third inside surface which extends in a direction that is generally perpendicular to the first inside surface and to the second inside surface. An outside engagement surface may comprise a first outside surface that is generally planar, a second outside surface that is generally planar and which extends in a direction that is generally perpendicular to the first outside surface and a third outside surface that is generally planar and which extends in a direction that is generally perpendicular to the first outside surface and to the second outside surface.

In another aspect of the invention, a sterilization wrap protector for a sterilization tray, that may separate the sterilization tray from a sterilization wrap, comprises a foam plastic material comprising a tray corner engagement surface. The tray corner engagement surface may comprise a first corner engagement surface that is generally planar, a second corner engagement surface that is generally planar and which extends in a direction that is generally perpendicular to the first corner engagement surface and a third corner engagement surface that is generally planar and which extends in a direction that is generally perpendicular to the first corner engagement surface and to the second corner engagement surface. The first, second and third corner engagement surfaces may intersect to define an inside corner point. A sterilization wrap engagement surface comprises a first sterilization wrap engagement surface that is generally planar, a second sterilization wrap engagement surface that is generally planar and which extends in a direction that is generally perpendicular to the first sterilization wrap engagement surface and a third sterilization wrap engagement surface that is generally planar and which extends in a direction that is generally perpendicular to the first sterilization wrap engagement surface and to the second sterilization wrap engagement surface. The first, second and third sterilization wrap engagement surfaces may intersect to define an outside corner point and the tray corner engagement surface and the sterilization wrap engagement surface may be separated by a generally constant thickness of the foam plastic material.

In accordance with a further aspect of the present invention, a sterilization wrap protector separates a sterilization tray from a sterilization wrap, the protector may comprise a foam plastic material including an inside engagement surface. The inside engagement surface may comprise a first inside surface that is generally planar, a second inside surface which extends from the first inside surface and that is generally arcuate in cross section and a third inside surface which also extends from the first inside surface and is also generally arcuate in cross section. An outside engagement surface may comprise a first outside surface that is generally planar, a second outside surface that is generally planar and which extends in a direction that is generally perpendicular to the first outside surface and a third outside surface that is generally planar and which extends in a direction that is generally perpendicular to the first outside surface and to the second outside surface.

### BRIEF DESCRIPTION OF THE DRAWINGS

Other features and advantages of the present invention will become apparent upon reading the following detailed description in conjunction with the accompanying drawings wherein like elements are numbered alike and wherein:

FIG. 1 is a perspective view of a prior art corner protector;

FIG. 2 is an enlarged, perspective view of the prior art corner protector of FIG. 1 shown attached to a sterilization tray;

FIG. 3 is a perspective view of a sterilization wrap corner protector in accordance with an embodiment of the present invention;

FIG. 4 is an exploded view, in perspective, of a plurality of sterilization wrap corner protectors of FIG. 1, a sterilization tray and a sterilization wrap; and

FIG. 5 is a perspective view of a sterilization wrap corner protector in accordance with another embodiment of the present invention.

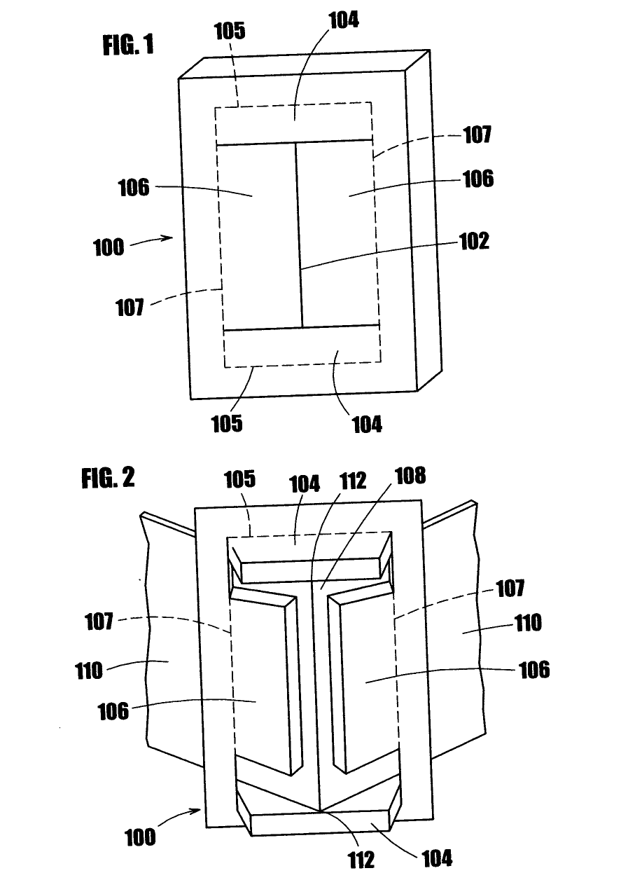

### DETAILED DESCRIPTION OF THE PREFERRED EMBODIMENT

In accordance with the present invention, a more effective corner protector, which better prevent rips and tears in a sterilization wrap and is more easily attached to a sterilization tray, is provided. In one embodiment, a corner protector is configured to completely cover each of the sharp corner edge points of a sterilization tray.

Referring now to FIG. 3, a corner protector, in accordance with an embodiment of the present invention is shown generally at 10. The corner protector 10 may comprise a plastic foam material that provides a suitable cushioning for preventing rips or tears in a sterilization wrap wrapped around a sterilization tray. The corner protector 10 may comprise a non-reticulated polyurethane foam having the physical properties provided in TABLE 1.

TABLE 1

| Physical Property | | |
|---|---|---|
| (Tested According | | |
| to ASTM D3574/86) | Minimum | Average |
| Density | 2.0 ± 10% lbs./cu.ft. | 2.0 ± 10% lbs./cu.ft. |
| Tensile Strength | 22 psi. | 27 psi. |
| Ultimate Elongation | 58% | 85% |
| Tear Resistance | 1.1 ppi. | 1.6 ppi. |
| Compression set, Cₜ, % | Max. 10% | Max. 10% |
| 50% Deflection | | |
| Compression Force | 2.4 psi. | 2.9 psi. |
| Deflection, 70% Deflection | | |
| Indentation Force Deflection, | 92 lbs. | 112 lbs. |
| lbs. 25% Deflection, 4 inches | | |
| thick | | |
| Cell Count (visual) pores per | 50 ± 5/in. | 50 ± 5/in. |
| inch | | |
| Retention of Tensile Strength | Min. 70% | Min. 70% |
| after 5 hours steam Autoclave | | |
| @ 120° C. | | |

The corner protector 10 comprises an inside or tray corner engagement surface 12 that, in turn, may comprise a first inside or corner engagement surface 14, a second inside or corner engagement surface 16 and a third inside or corner engagement surface 18. The first corner engagement surface 14 may be generally planar and may be bound, at one side by an upper surface 20. The second corner engagement surface 16 may also be generally planar and extends in a direction that is generally perpendicular to the first corner engagement surface 14. The third corner engagement surface 18 further may be generally planar and extends in a direction that is generally perpendicular to both the first corner engagement surface 14 and to the second corner engagement surface 16. As illustrated, the first and second corner engagement surfaces 14 and 16 may have a surface area that is substantially less than that of the third corner engagement surface 18. An inside corner point 22 may be located at the intersection of the first, second and third corner engagement surfaces 14, 16 and 18.

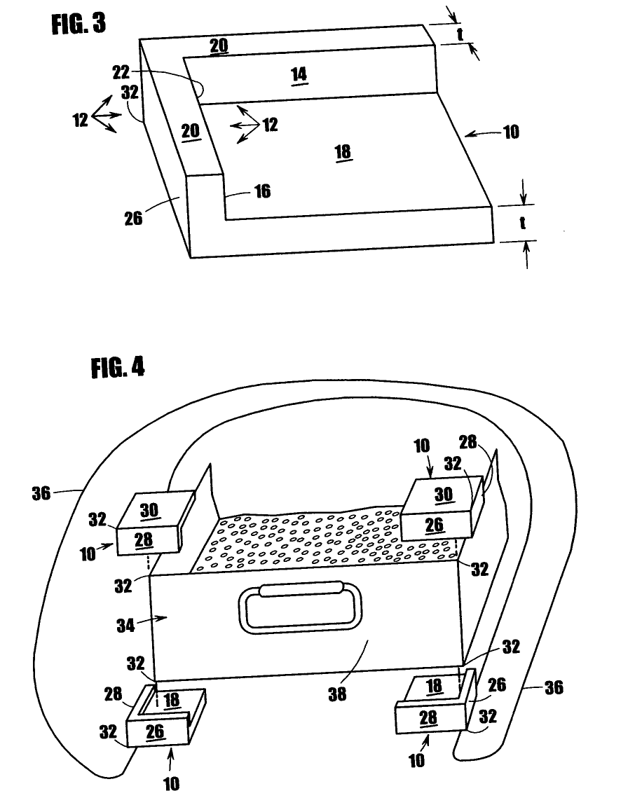

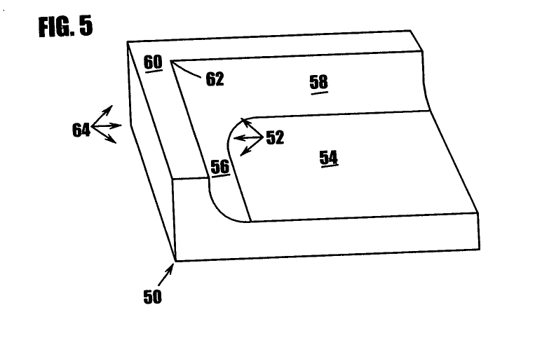

Referring now also to FIG. 4, the corner protector 10 may also comprise an outside or sterilization wrap engagement surface 24 that, in turn, may comprise a first outside or sterilization wrap engagement surface 26, a second outside or sterilization wrap engagement surface 28 and a third outside or sterilization wrap engagement surface 30. The first sterilization wrap engagement surface 26 may be generally planar as may be the second sterilization wrap engagement surface 28 which may extend in a direction that is generally perpendicular to the first sterilization wrap engagement surface. The third sterilization wrap engagement surface 30 may further be generally planar and may extend in a direction that is generally perpendicular to the first sterilization wrap engagement surface 26 and to the second sterilization wrap engagement surface 28. As shown, the first and second sterilization wrap engagement surfaces 26 and 28 may have a surface area that is substantially less than that of the third sterilization wrap engagement surface 30. The first, second and third sterilization wrap engagement surfaces 26, 28 and 30 intersect to define an outside corner point 32.

Preferably, although not necessarily, a generally constant thickness (t) of the foam plastic material separates the tray corner engagement surface 12 and the sterilization wrap engagement surface 24.

As shown in FIG. 4, the corner protector 10 may be attached to each corner point 32 of a sterilization tray 34 so that the inside corner 22 of the corner protector engages a corner point 32 of the sterilization tray. A sterilization wrap 36 may then be wrapped about the sterilization tray 34 in a known manner. It will be appreciated that rather than assembling a corner protector 10 to all four corner points of an end 38 of the sterilization tray 34, only two corners may be protected where, for example, a cover (not shown) is employed with the sterilization tray.

Referrring now to FIG. 5, another embodiment of a corner protector in accordance with the present invention is shown generally at 50. The corner protector 50 may also comprise a plastic foam material that provides a suitable cushioning and may comprise a reticulated polyurethane foam that has a foam grade of 2000 yellow. The corner protector 50 may also have the physical properties provided in TABLE 2, although, it will be understood that a material having the properties of TABLE 1 may be employed as well as any other suitably flexible and readily moldable material.

TABLE 2

| Test or Standard | Specification |
|---|---|
| Density | 1.90 ± .05 lbs/cu.ft. |
| IFD on 4″ at 25% Deflection | 90-105 |
| Resilience - % Minimum | .35 |
| Tensile Strength - PSI Minimum | 17 |
| Elongation - % Minimum | 120 |
| Tear Strength lb/in, Minimum | 2.0 |
| 90% Compression Set - % | 15.0 |
| Maximum | |

The corner protector 50 comprises an inside or tray corner engagement surface 52 that, in turn, may comprise a first inside or corner engagement surface 54, a second inside or corner engagement surface 56 and a third inside or corner engagement surface 58. The first corner engagement surface 54 may be generally planar and may be bound at one side by an upper surface 60. The second corner engagement surface 56 may be generally arcuate in cross section and may extend from the first corner engagement surface 54. The third corner engagement surface 58 also may be generally arcuate in cross section and also may extend from the first corner engagement surface 54. At least a portion of each of the second and third engagement surfaces may intersect at 62 to form an angle of approximately ninety degrees therebetween.

The corner protector 50 may also comprise an outside or sterilization wrap engagement surface 64 that may be similar to that described above in connection with FIG. 4 and thus reference may be had above for further details of the sterilization wrap engagement surface. Also, a corner protector 50 may be fitted to each corner point 32 of a sterilization tray 34 similar to that also described above in connection with FIG. 4.

The principles, preferred embodiments and modes of operation of the presently disclosed corner protectors have been described in the foregoing specification. However, the present invention is not to be construed as limited to the particular embodiments shown, as these embodiments are regarded as illustrious rather than restrictive. Moreover, variations and changes may be made by those skilled in the art without departing from the spirit of the presently disclosed corner protector. For example, while the embodiments of the corner protector described above are described as being employed solely between a sterilization tray and a sterilization wrap, it will be understood that the corner protector may be employed with additional materials and/or between other materials or devices. Accordingly, terms such as tray corner engagement surface and sterilization wrap engagement surface are employed herein for ease in describing disclosed embodiments of the present invention.

1. A sterilization wrap protector for a sterilization tray, the sterilization wrap protector separating the sterilization tray from a sterilization wrap, the protector comprising:
a material comprising:
an inside engagement surface comprising a first inside surface that is generally planar, a second inside surface that is generally planar and which extends in a direction that is generally perpendicular to the first inside surface and a third inside surface which extends in a direction that is generally perpendicular to the first inside surface and to the second inside surface; and
an outside engagement surface comprising a first outside surface that is generally planar, a second outside surface that is generally planar and which extends in a direction that is generally perpendicular to the first outside surface and a third outside surface that is generally planar and which extends in a direction that is generally perpendicular to the first outside surface and to the second outside surface.

2. The protector of claim 1, wherein the inside engagement surface and the outside engagement surface are separated by a generally constant thickness of the material.

3. The protector of claim 2, wherein the material comprises a foam plastic material.

4. The protector of claim 1, wherein the first and second inside surfaces have a surface area that is substantially less than that of the third inside surface.

5. The protector of claim 1, wherein the first and second outside surfaces have a surface area that is substantially less than that of the third outside surface.

6. The protector of claim 1, wherein the sterilization tray comprises a sterilization tray side surface, a sterilization tray front surface and a sterilization tray bottom surface and wherein each of the first, second and third inside surfaces each have a surface area that is substantially less than the surface area of each of the sterilization tray side surface, the sterilization tray front surface and the sterilization tray bottom surface, respectively.

7. The protector of claim 1, wherein the third outside engagement surface is generally planar.

8. The protector of claim 1, wherein the first, second and third inside surfaces intersect to define an inside corner point and wherein the first, second and third outside surfaces intersect to define an outside corner point.

9. The protector of claim 1, wherein the first outside surface and the second outside surface intersect at an angle which is approximately ninety degrees, wherein the first outside surface and the third outside surface intersect at an angle which is approximately ninety degrees and wherein the second outside surface and the third outside surface intersect at an angle which is approximately ninety degrees.

10. A sterilization wrap protector for a sterilization tray, the sterilization wrap protector separating the sterilization tray from a sterilization wrap, the protector comprising:
a material comprising:
a tray corner engagement surface comprising a first corner engagements surface that is generally planar, a second corner engagement surface that is generally planar and which extends in a direction that is generally perpendicular to the first corner engagement surface and a third corner engagement surface that is generally planar and which extends in a direction that is generally perpendicular to the first corner engagement surface and to the second corner engagement surface wherein the first, second and third corner engagement surfaces intersect to define an inside corner point; and
a sterilization wrap engagement surface comprising a first sterilization wrap engagement surface that is generally planar, a second sterilization wrap engagement surface that is generally planar and which extends in a direction that is generally perpendicular to the first sterilization wrap engagement surface and a third sterilization wrap engagement surface that is generally planar and which extends in a direction that is generally perpendicular to the first sterilization wrap engagement surface and to the second sterilization wrap engagement surface wherein the first, second and third sterilization wrap engagement surfaces intersect to define an outside corner point;
wherein the tray corner engagement surface and the sterilization wrap engagement surface are separated by a generally constant thickness of the material.

11. The protector of claim 10, wherein the material comprises a foam plastic material.

12. The protector of claim 10, wherein the first and second corner engagement surfaces have a surface area that is substantially less than that of the third corner engagement surface.

13. The protector of claim 10, wherein the first and second sterilization wrap engagement surfaces have a surface area that is substantially less than that of the third sterilization wrap engagement surface.

14. The protector of claim 10, wherein the sterilization tray comprises a sterilization tray side surface, a sterilization tray front surface and a sterilization tray bottom surface and wherein each of the first, second and third corner engagement surfaces each have a surface area that is substantially less than that of the sterilization tray side surface, the sterilization tray front surface and the sterilization tray bottom surface, respectively.

15. A sterilization wrap protector for a sterilization tray, the sterilization wrap protector separating the sterilization tray from a sterilization wrap, the protector comprising:
a material comprising:
a tray corner engagement surface comprising a first corner engagement surface that is generally planar, a second corner engagement surface that is generally planar and which extends in a direction that is generally perpendicular to the first corner engagement surface and a third corner engagement surface that is generally planar and which extends in a direction that is generally perpendicular to the first corner engagement surface and to the second corner engagement surface wherein the first, second and third corner engagement surfaces intersect to define an inside corner point; and
a sterilization wrap engagement surface comprising a first sterilization wrap engagement surface that is generally planar, a second sterilization wrap engagement surface that is generally planar and which extends in a direction that is generally perpendicular to the first sterilization wrap engagement surface and a third sterilization wrap engagement surface that is generally planar and which extends in a direction that is generally perpendicular to the first sterilization wrap engagement surface and to the second sterilization wrap engagement surface wherein the first, second and third sterilization wrap engagement surfaces intersect to define an outside corner point;
wherein the tray corner engagement surface and the sterilization wrap engagement surface are separated by a generally constant thickness of the material, the first and second corner engagement surfaces have a surface area that is substantially less than that of the third corner engagement surface, the first and second sterilization wrap engagement surfaces have a surface area that is substantially less than that of the third sterilization wrap engagement surface and wherein the sterilization tray comprises a sterilization tray side surface, a sterilization tray front surface and a sterilization tray bottom surface and wherein each of the first, second and third corner engagement surfaces each have a surface area that is substantially less than that of the sterilization tray side surface, the sterilization tray front surface and the sterilization tray bottom surface, respectively.

16. The protector of claim 15, wherein the material comprises a foam plastic material.

17. A sterilization wrap protector for a sterilization tray, the sterilization wrap protector separating the sterilization tray from a sterilization wrap, the protector comprising:
a material comprising:
an inside engagement surface comprising a first inside surface that is 0s generally planar, a second inside surface which extends from the first inside surface and that is generally arcuate in cross section and a third inside surface which also extends from the first inside surface and is also generally arcuate in cross section; and
an outside engagement surface comprising a first outside surface that is generally planar, a second outside surface that is generally planar and which extends in a direction that is generally perpendicular to the first outside surface and a third outside surface that is generally planar and which extends in a direction that is generally perpendicular to the first outside surface and to the second outside surface.

18. The protector of claim 17, wherein at least a portion of the second inside surface and at least a portion of the third inside surface intersect at an angle which is approximately ninety degrees.

19. The protector of claim 17, wherein the first outside surface and the second outside surface intersect at an angle which is approximately ninety degrees, wherein the first outside surface and the third outside surface intersect at an angle which is approximately ninety degrees and wherein the second outside surface and the third outside surface intersect at an angle which is approximately ninety degrees.

20. The protector of claim 17, wherein the material comprises a foam plastic material.

21. The protector of claim 20, wherein the foam plastic material is a reticulated polyurethane foam.

22. The protector of claim 3, wherein the foam plastic material is a reticulated polyurethane foam.

23. The protector of claim 11, wherein the foam plastic material is a reticulated polyurethane foam.

24. The protector of claim 16, wherein the foam plastic material is a reticulated polyurethane foam.
